(19) United States
(12) Patent Application Publication
(10) Pub. No.: US 2016/0302684 A1
(43) Pub. Date: Oct. 20, 2016

(54) ELECTRODE HOUSING AND A MONITORING DEVICE

(71) Applicant: LifeWatch Technologies, LTD., Rehovot (IL)

(72) Inventors: Asher en-Haim, Bat Yam (IL); Yossi Lovton, Nes Zeyona (IL)

(21) Appl. No.: 14/685,866

(22) Filed: Apr. 14, 2015

(51) Int. Cl. A61B 5/0408; A61B 5/00; A61B 5/04; H05K 5/02

(52) U.S. Cl. CPC A61B 5/04085; H05K 5/0217; A61B 5/0006; A61B 5/04012

(57) ABSTRACT

An electrode housing that is made of an elastic material and includes a trench that includes an exterior sidewall, a bottom and an internal sidewall; a group of structural elements; wherein the internal sidewall is coupled to the group of structural elements; wherein the group of structural elements is surrounded by the trench and defines a cavity that matches a shape and size of an electrode.

### BACKGROUND OF THE INVENTION

During the last couple of years the importance of health monitoring has increased. Remote health monitoring allows the persons to continue with their daily routine without being restricted to hospitals of dedicated laboratories.

There is a growing need to provide efficient health monitors that are compact and are accurate.

There is also a growing need to provide a monitoring device installation method that can be executed by a person and allow the person to position the monitoring device at a desired orientation.

There is also a growing need to provide a robust and noise reducing electrode housing for electrodes of the monitoring device.

### SUMMARY OF THE INVENTION

According to various embodiments of the invention there is provided a monitoring device that may include a detachable processing and transmitting module, multiple electrodes for sensing electrocardiogram signals; an elastic layer that comprises multiple electrode housings for supporting the multiple electrodes; wherein each electrode housing that is made of an elastic material and comprises a trench that includes an exterior sidewall, a bottom and an internal sidewall; a group of structural elements; wherein the internal sidewall is coupled to the group of structural elements; wherein the group of structural elements is surrounded by the trench and defines a cavity that matches a shape and size of an electrode.

According to various embodiments of the invention there is provided an electrode housing that is made of an elastic material and may include a trench that includes an exterior sidewall, a bottom and an internal sidewall; a group of structural elements; wherein the internal sidewall is coupled to the group of structural elements; wherein the group of structural elements is surrounded by the trench and defines a cavity that matches a shape and size of an electrode. The trench and the group of structural elements may be integrated.

The depth of the trench may be few millimeters.

The trench and the group of structural elements may be radially symmetric.

The group of structural elements may include an annular segmented base and an inner annular cover that define the cavity.

The group of structural elements may include an external ring and an inner trench that is delimited between the inner annular cover and the external ring.

The annular segmented base may define an opening that is shaped and sized to match an upper part of an electrode.

The elastic material can be thermoplastic polyurethane.

### BRIEF DESCRIPTION OF THE DRAWINGS

The foregoing and other objects, features, and advantages of the present invention will become more apparent from the following detailed description when taken in conjunction with the accompanying drawings. In the drawings, similar reference characters denote similar elements throughout the different views, in which:

FIG. 1 illustrates a person that wears a monitoring device positioned at a first orientation on the chest of the person according to an embodiment of the invention;

FIG. 2 illustrates a person that wears a monitoring device positioned at a second orientation on the chest of the person according to an embodiment of the invention;

FIG. 3 illustrates a person that wears a monitoring device positioned at a third orientation on the chest of the person according to an embodiment of the invention;

FIG. 4 illustrates a person that wears a monitoring device positioned at a fourth orientation on the chest of the person according to an embodiment of the invention;

FIG. 5 illustrates a person that wears a monitoring device positioned at a fifth orientation on the chest of the person according to an embodiment of the invention;

FIG. 6 illustrates a person that wears a monitoring device positioned at a lower chest and additional electrodes that are coupled to the monitoring device according to an embodiment of the invention;

FIG. 7 is a flow chart of a method according to an embodiment of the invention;

FIG. 8 illustrates a person that wears a monitoring device, a computerized control unit, a cellular network base station and a remote monitoring system according to an embodiment of the invention;

FIG. 9 is a screenshot of a display of the computerized control unit according to an embodiment of the invention;

FIG. 10 is a flow chart of a method according to an embodiment of the invention;

FIG. 11 is a flow chart of a method according to an embodiment of the invention;

FIG. 12 illustrates a top part of the housing of the monitoring device according to an embodiment of the invention;

FIG. 13 is an exploded view of the monitoring device according to an embodiment of the invention;

FIG. 14 is a block diagram of the monitoring device according to an embodiment of the invention;

FIG. 15 illustrates the monitoring device with additional electrodes according to an embodiment of the invention;

FIG. 16 illustrates an elastic layer of the monitoring device according to an embodiment of the invention;

FIG. 17 illustrates a portion of an elastic layer of the monitoring device according to an embodiment of the invention;

FIG. 18 is a cross sectional view of a portion of an elastic layer of the monitoring device according to an embodiment of the invention; and

FIG. 19 is a cross sectional view of a portion of an elastic layer of the monitoring device, of an electrode and other components of the monitoring device according to an embodiment of the invention.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

### Alignment of Monitoring Device to Electrical Heart Axis

According to an embodiment of the invention there is provided a method, computer readable medium and a computerized monitoring unit that assist in the alignment of the monitoring device to the electrical heart axis.

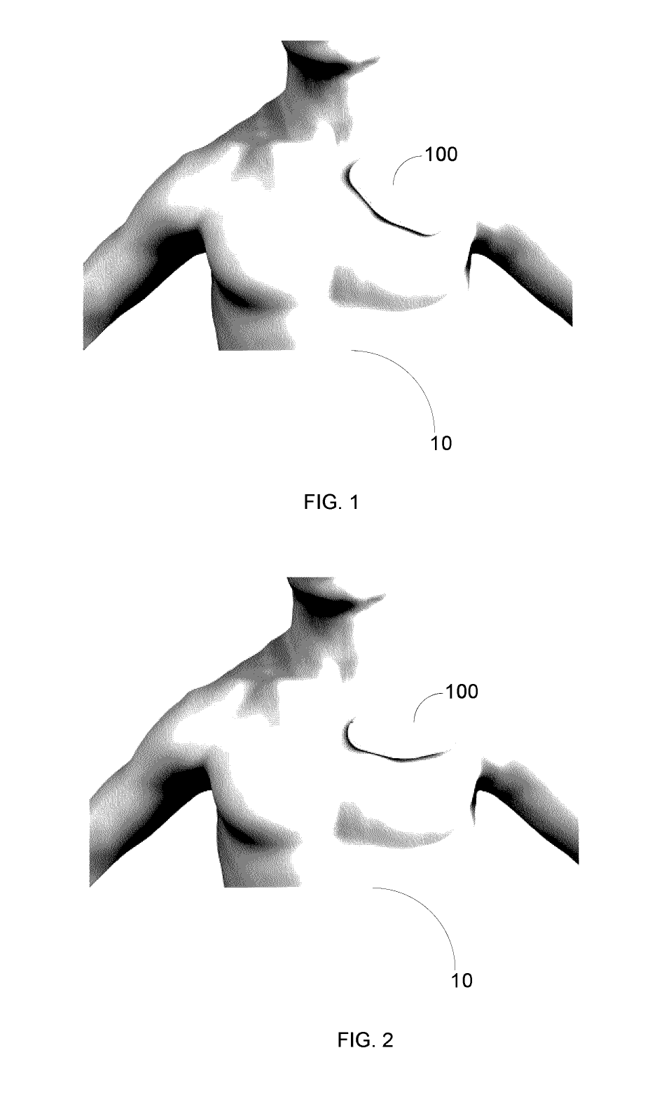

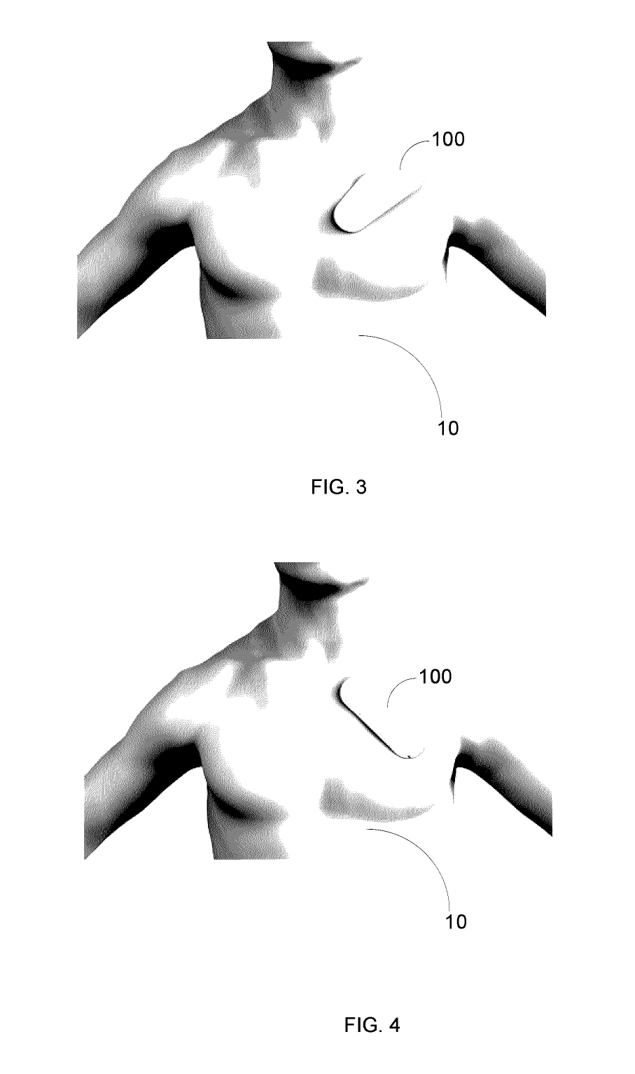

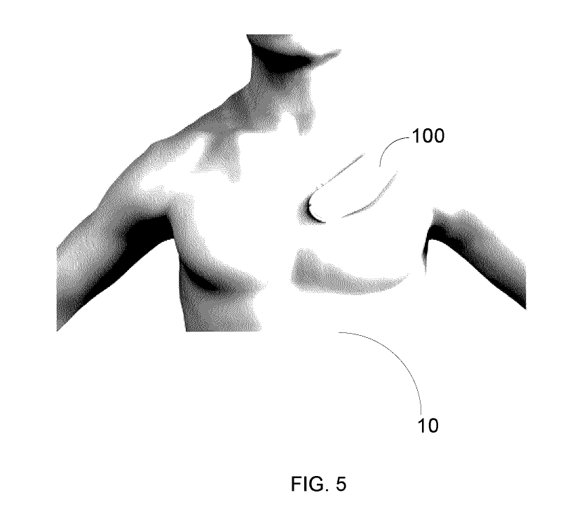

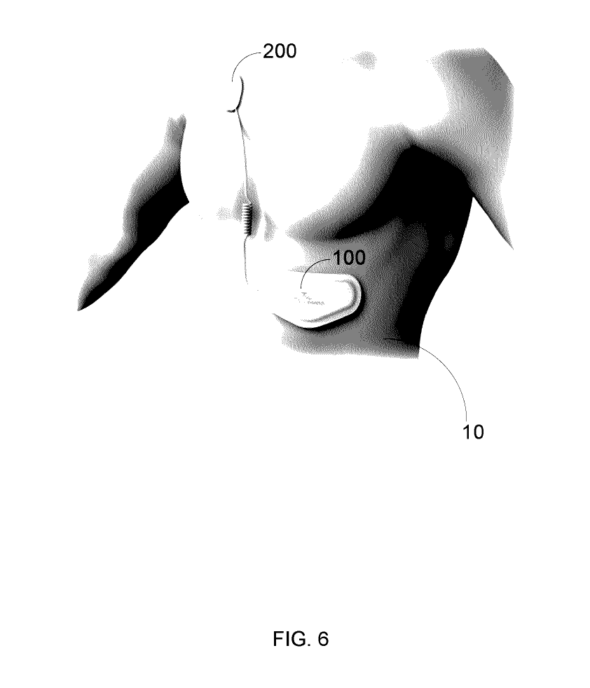

The alignment process includes multiple alignment iterations. FIGS. 1-5 illustrate an alignment process that includes five alignment iterations. The five alignment iterations form a first set of alignment iterations. If all the alignment iterations of the first alignment iterations fail (an adequate alignment was not obtained) then the first set of alignment iterations is followed by attaching additional electrodes and positioning the monitoring device at the lower chest of the person.

It is noted that the number of alignment iterations may differ from five—may exceed five or be less than five.

It is noted that the rotations of the monitoring device between adjacent alignment iterations may be substantially even or may differ from each other.

It is noted that the alignment process may execute a certain amount of alignment iterations regardless of the results of the alignment iterations and then determine a selected position of the monitoring device.

It is noted that the alignment process may stop the execution of the alignment iterations when finding an alignment. For example—if in the third alignment iteration of the five alignment iterations of FIG. 1-5 an alignment is found than the alignment process may stop and not proceed to the fourth and fifth alignment iterations.

It is further noted that the iteration process may include a conversion phase during which one or more orientations are evaluated, wherein the angular differences between evaluated orientations of the convergence phase are smaller than the angular differences between orientations of alignment iterations that preceded the convergence phase.

It is further notes that any change of the orientation of the monitoring device may also include a non-rotational change in the position of the monitoring device.

FIG. 1 illustrates a person 10 that wears monitoring device 100 positioned at

According to an embodiment of the invention, monitoring device 100 is a compact mobile electrocardiographic system, which is conveniently either handily portable by a medical practitioner or a first aid provider, in order to quickly diagnose a person, or adapted to be carried by a person in potential hazard for longer periods of time, either for analysis or for monitoring his condition. Being able to transport the data wirelessly facilitate continuing monitoring by a medical center.

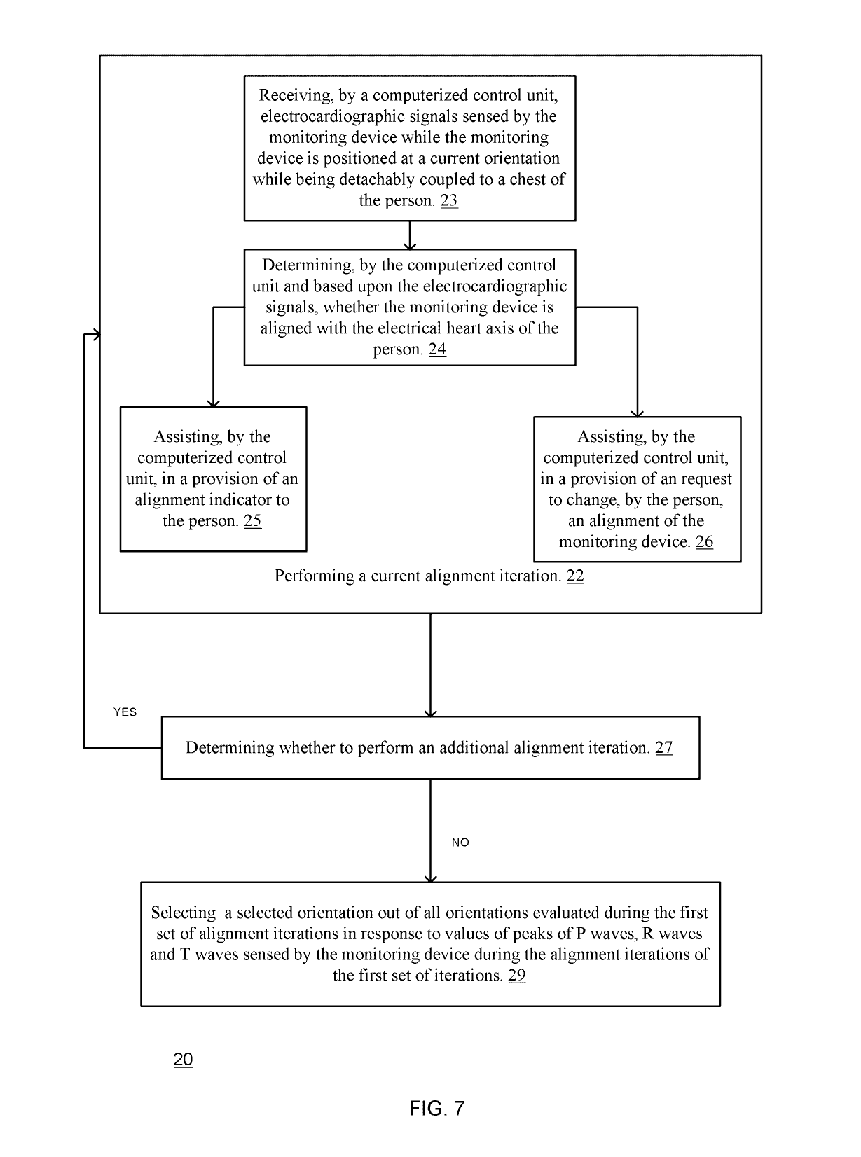

FIG. 7 illustrates method 20 according to an embodiment of the invention.

Method 20 starts by step 22 of performing a current alignment iteration.

Step 22 may include steps 23, 24, 25, and 26.

Step 23 may include receiving, by a computerized control unit, electrocardiographic signals sensed by the monitoring device while the monitoring device is positioned at a current orientation while being detachably coupled to a chest of the person.

Step 23 may be followed by step 24 of determining, by the computerized control unit and based upon the electrocardiographic signals, whether the monitoring device is aligned with the electrical heart axis of the person.

Step 24 of determining whether the monitoring device is aligned with the electrical heart axis may include detecting P waves, R waves and T waves.

Step 24 of determining whether the monitoring device is aligned with the electrical heart axis may be responsive to amplitudes of a P wave sensed by the monitoring device and a ratio between amplitudes of a T wave sensed by the monitoring device and an R wave sensed by the monitoring device. An amplitude of a wave (R wave, T wave P wave) may include a peak of the wave, an average of the wave or an outcome of any other mathematical function applied on amplitudes of the wave during one or more points in time.

Step 24 may include determining that the monitoring device is aligned with the electrical hear when a peak of a P wave sensed by the monitoring device exceed an amplitude threshold and a ratio between a peak of a T wave sensed by the monitoring device and a peak of a R wave sensed by the monitoring device exceeds a ratio threshold.

If determining that the monitoring device is aligned with the electrical heart then step 24 is followed by step 25 of assisting, by the computerized control unit, in a provision of an alignment indicator to the person.

The assisting in a provision of the alignment indicator may include generating an audio indicator, generating a visual indicator, triggering the monitoring device to generate the alignment indicator, and the like.

If determining that the monitoring device is misaligned with the electrical heart axis then step 24 is followed by step 26 of assisting, by the computerized control unit, in a provision of an request to change, by the person, an alignment of the monitoring device.

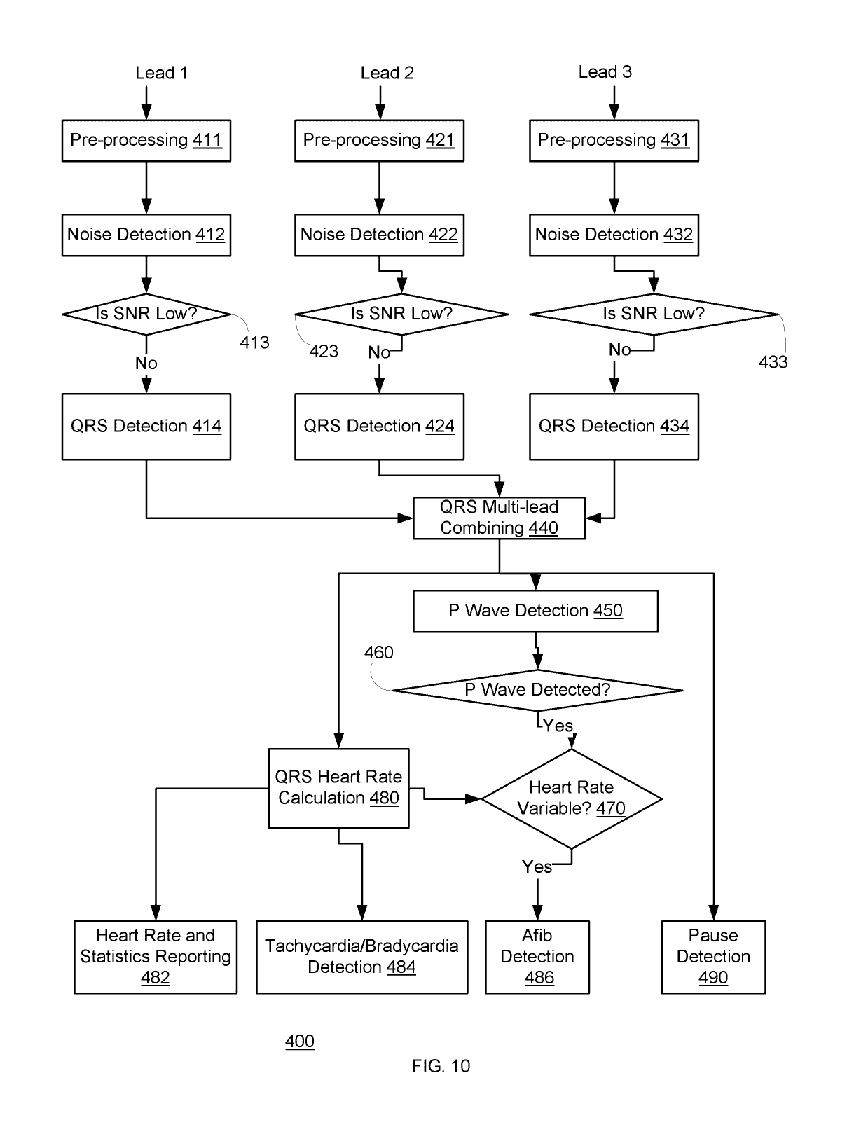

The request may include an indication about a desired change of orientation of the monitoring device. FIG. 10 illustrates a screenshot of a display of a computerized control unit that displays to the person either the current position of the monitoring device or a requested new position of the monitoring device.

The assisting in a provision of the request to change the alignment of the device may include generating an audio indicator, generating a visual indicator, triggering the monitoring device to generate the alignment indicator, and the like.

Step 22 may be followed by step 27 of determining whether to perform an additional alignment iteration.

Step 27 may include determining to perform another alignment iteration if, for example, the predefined amount of alignment iterations within a first set of alignment iteration was not reached yet.

Step 27 may include determining to perform another alignment iteration if, for example, the predefined amount of alignment iterations within a first set of alignment iteration was not reached yet and neither one of the alignment iterations that were executed achieved an alignment between the monitoring device and the electrical heart axis.

Step 27 may include determining not to perform another alignment iteration when the first set of alignment iterations failed (no alignment) and jump to step 30 of assisting in a provision of a request to position the monitoring at a certain position, attach additional electrodes to the person and couple the additional electrodes to the monitoring device.

Step 27 may include determining not to perform another alignment iteration—when the one of the alignment iterations succeeded. This can be the last alignment iteration that was executed or one of the previously execute alignment iterations.

Step 27 may include determining not to perform another alignment iteration and jumping to step 29 of selecting a selected orientation out of all orientations evaluated during the first set of alignment iterations in response to values of peaks of P waves, R waves and T waves sensed by the monitoring device during the alignment iterations of the first set of iterations. Step 29 may include assisting, by the computerized control unit, in a provision of an request to change, by the person, an alignment of the monitoring device to the selected orientation—if the selected alignment differs from the current alignment of the monitoring device.

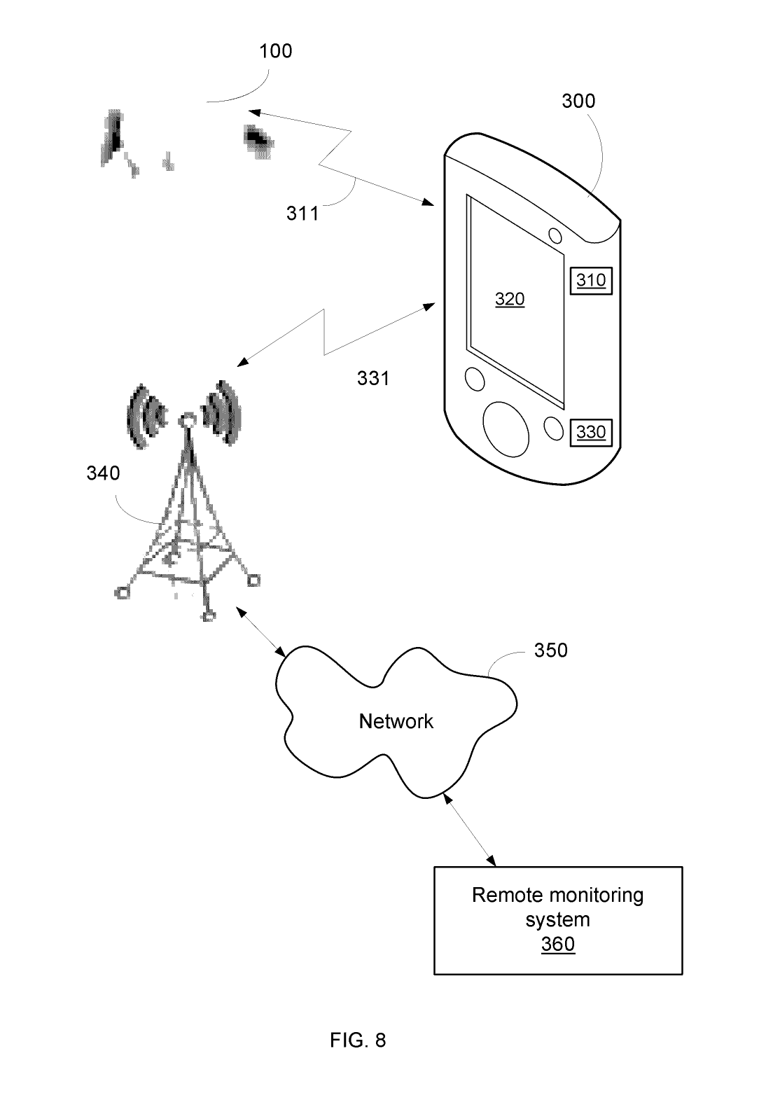

FIG. 8 illustrates a monitoring device 100 that is detachably attached to a person 10, a computerized control unit 300, a cellular base station 340, a network 350 and a remote monitoring system 360 according to an embodiment of the invention.

The computerized control unit 300 wirelessly communicates via a wireless link 311 with monitoring device 100. The computerized control unit 300 wirelessly communicates with a remote monitoring system 360 via wireless link 331, cellular base station 340 and network 350. Network 350 may be the Internet or any other wired and/or wireless network.

It is assumed that wireless link 311 is a short range communication link and that wireless link 331 is a long range communication link, but this is not necessarily so. Any type of communication links and/or communication protocols may be used to convey information between monitoring device 100, computerized control unit 300 and remote monitoring system 360.

The computerized control unit 300 may be a personal data accessory, a mobile phone, a laptop computer, a desktop computer, a tablet, a media player and the like. It is capable of executing method 20.

According to an embodiment of the invention the computerized control unit 300 includes a transceiver 310, a man machine interface 320 and an electrocardiographic processor 330. The electrocardiographic processor 330 may be any type of hardware processor that is configured to execute instructions for processing electrocardiographic signals sensed by monitoring device 100.

The man machine interface may be a display, a touch screen, an audio generator, one or more control buttons or any other man machine interface known in the art.

The computerized control unit 300 may be configured to perform a first set of alignment iterations for aligning a monitoring device and an electrical heart axis of the person.

Each alignment iteration of the first set may include: (I) receiving, by the transceiver 310, electrocardiographic signals sensed by the monitoring device while the monitoring device is positioned at certain orientation while being detachably coupled to a chest of the person; (II) determining, by the electrocardiographic processor 330 and based upon the electrocardiographic signals, whether the monitoring device is aligned with the electrical heart axis of the person; (III) wherein when determining that the monitoring device is aligned with the electrical heart then assisting, by at least one of the man machine interface 320 or the transceiver 310, in a provision of an alignment indicator to the person; (IV) wherein when determining that the monitoring device is misaligned with the electrical heart axis then assisting, by at least one of the man machine interface 320 or the transceiver 310, in a provision of an request to change, by the person, an alignment of the monitoring device.

The electrocardiographic processor 330 may be configured to receive electrocardiogram (ECG) data from the monitoring device 100 and to detect events such as but not limited to heart pause, Arial Fibrillation, Tachycardia and Bradycardia. An occurrence of an event can be transmitted to the remote monitoring system 360. The remote monitoring system 360 may be staffed by medical personnel.

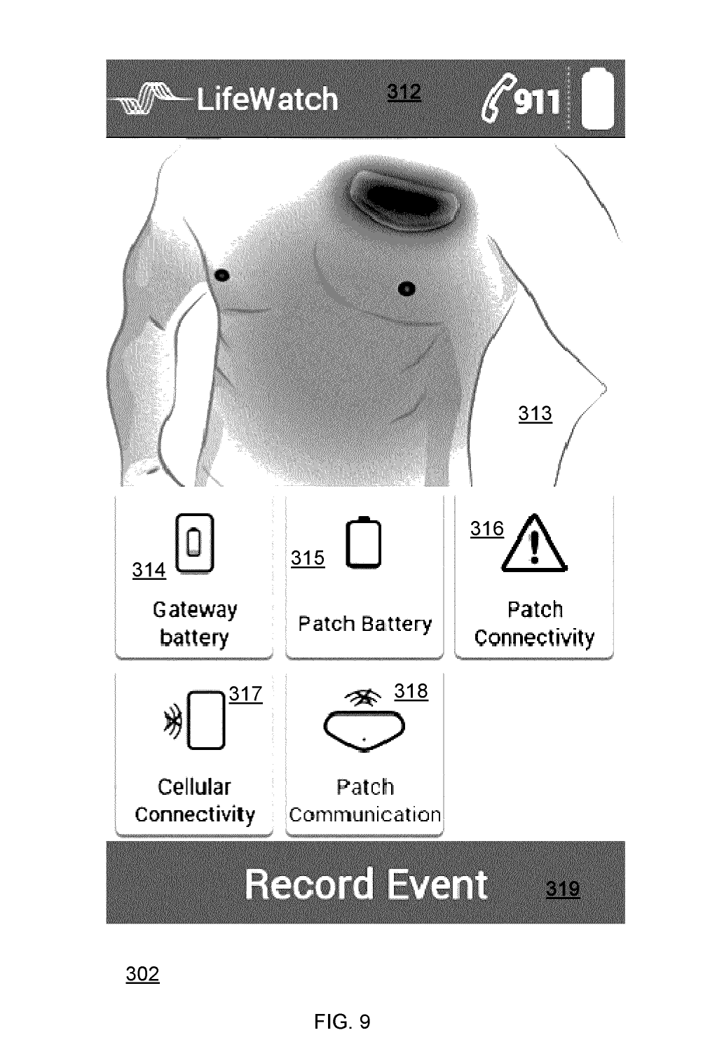

FIG. 9 is a screenshot 302 of a display of the computerized control unit 300 according to an embodiment of the invention.

The screenshot 302 includes the following segments: (i) a top segment 312 that is illustrates as including a quick link to an emergency call “911”, (ii) a main segment 313 that illustrates the torso of the person and a current (estimated) position of the monitoring device or a next (requested) position of the monitoring device, wherein an icon that represents the monitoring device may indicate whether the displayed location is the current location or the next location, (iii) status segments indicative of a status of a battery of the computerized control unit (“gateway battery” segment 314), of a status of a battery of the monitoring device (“patch battery” segment 315), of a status of connectivity (contact between person and electrodes) of the monitoring device (“patch connectivity” segment 316) that may indicate about the manner in which the monitoring device is coupled to the person, status of communication between the computerized control unit and the mobile base station (“cellular connectivity” segment 317), status of communication between the monitoring device and the computerized control unit (“patch communication” segment 319).

The arrangement of the screen shot, the events that are reported on the display may differ from those illustrated in FIG. 9.

FIG. 10 illustrates a method 400 according to an embodiment of the invention.

The software application installed in the computerized control unit 300 analyzes the ECG data from the three electrodes 131 for cardiac arrhythmias.

If an arrhythmia is detected, the computerized control unit 300 transmits a physiological alarm to the remote monitoring system 360.

The transmitted information may include the cardiac arrhythmia type, event initiation timestamp, raw ECG data, and event termination timestamp.

In addition to analyzing the data and sending arrhythmia events, the computerized control unit 300 will have an option to continuously send the recorded ECG data files to the remote monitoring system 360 in a later phase.

The computerized control unit 300 may analyze the ECG data for cardiac arrhythmias. If an arrhythmia is detected, the computerized control unit 300 transmits a physiological alarm to the remote monitoring system. The transmitted information may include includes the cardiac arrhythmia type, event initiation timestamp, raw ECG data, and event termination timestamp. In addition to analyzing the data and sending arrhythmia events, the system will have an option to continuously send the recorded ECG data files to the Monitoring Center in a later phase.

The computerized control unit 300 can detect cardiac arrhythmia events such as Pause, Atrial Fibrillation, Tachycardia, and Bradycardia (according to the device trigger settings).

The computerized control unit 300 can perform the analysis using parameters that can be modified by a qualified technician, within an allowable range per request.

The ECG signals from the first, second and third electrodes are processes by three corresponding sequences of steps.

The ECG signals from the first electrode are processed by steps 411 of pre-processing, 412 of noise detection, 413 of checking if the SNR is low and step 414 of QRS detection.

The ECG signals from the second electrode are processed by steps 421 of pre-processing, 422 of noise detection, 423 of checking if the SNR is low and step 424 of QRS detection.

The ECG signals from the third electrode are processed by steps 431 of pre-processing, 432 of noise detection, 433 of checking if the SNR is low and step 434 of QRS detection.

Steps 414, 424 and 434 of QRS detection are followed by step 440 of QRS multi-lead combining for merging the information obtained from the three electrodes.

Step 440 may be followed by step 450 of P wave detection, by step 490 of pause detection and step 480 of QRS heart rate calculation.

The outcome of step 480 may be fed to steps 482 of heart rate and statistics reporting, step 484 of Tachycardia and/or Bradycardia detection and step 470 is checking if the heat rate is variable. If so then step 470 is followed by step 486 of Atrial Fibrillation (Afib) detection.

Accordingly—method 400 may detect Pause, Tachycardia and Bradycardia based on QRS analysis and detect Atrial Fibrillation based on P Wave detection.

Step 490 of pause detection checks if the heart ceases to beat for more than a pre-defined time. Step 490 may measure the time following a QRS in which no new QRS was detected. When a QRS is detected, this step may ensure that confidence in the QRS is high. If confidence is high, the step resets and restarts a timer. When the timer reaches a pre-defined threshold, a pause event is detected.

Step 484 detects tachycardia when the heart rate exceeds a programmable value (Theart_rate_threshold) for a minimum threshold time.

Step 484 detects bradycardia when the heart rate is below a programmable value (Bheart_rate_threshold) for a minimum threshold time.

Step 486 may be detected when RR intervals (duration between successive beats) are of variable length and an absence of P-waves.

Steps 411, 421 and 431 of pre-processing may include drift and soft noise removal. The power supply of the computerized control unit may cause a ubiquitous electrical interference at the frequency of 50/60 Hz. The pre-processing may eliminate that interference by using a notch filter. Human respiration also induces interference at very low frequency. This interference may be removed by the pro-processing by applying a high pass filter with a cut-off at approximately 0.05 Hz. The ECG signal can be corrupted by electromyography (EMG), or a muscle contraction wave, which is generated during various body movements. In the ECG Analysis Algorithm, this type of noise is reduced with a low pass filter that removes the EMG, but has minimal effect on the ECG signal.

Steps 412, 422 and 432 may include strong noise detection—the noise is detected by using the distribution of the first and second derivatives of the ECG signals. The patterns that are identified as strong noise are marked and ignored in the QRS detection phase.

Steps 414, 424 and 434 of QRS Detection may include extracting and marking QRS positions from the ECG signals. The detection may be based on the morphological characteristic of the QRS. QRS usually has a higher slope compared to other signals in the ECG.

Step 440 may include combined data from the three electrodes to arrive at a single R timing value for the QRS. The morphology of the QRS extracted from each electrode may be analyzed for signal quality. The signals from each electrode may be weighted based on the signal quality and combined to provide a single value for the R timing.

Step 480 of QRS heart rate calculation may calculate the average heart rate in a sliding window of a pre-defined length to decrease the effect of noise on the QRS timing. The time between Rs in the QRS complexes, denoted as RR, is measured. The average value for the RRs in the sliding window is the calculated QRS heart rate. The standard deviation between RR lengths is also calculated.

Step 460 of detecting P waves may include detecting the contraction of the atria, denoted as a P wave. P waves may be detected by locating local maxima and minima preceding QRS complexes. To ensure the detection of the P wave, this step may search for a series of similar waves located at the same distance from the QRS complex. Additionally, these P waves should have the same average interval as the corresponding RRs.

Step 486 may include detecting atrial fibrillation by the presence of an irregular heart rate and the absence of P waves. Weighted values are assigned to irregular heart rate and absence of P waves based on the confidence level in the results from the QRS heart rate calculation and P Wave detection modules. The weighted values are then combined to determine the atrial fibrillation grade. If the grade exceeds a pre-defined threshold, atrial fibrillation is detected.

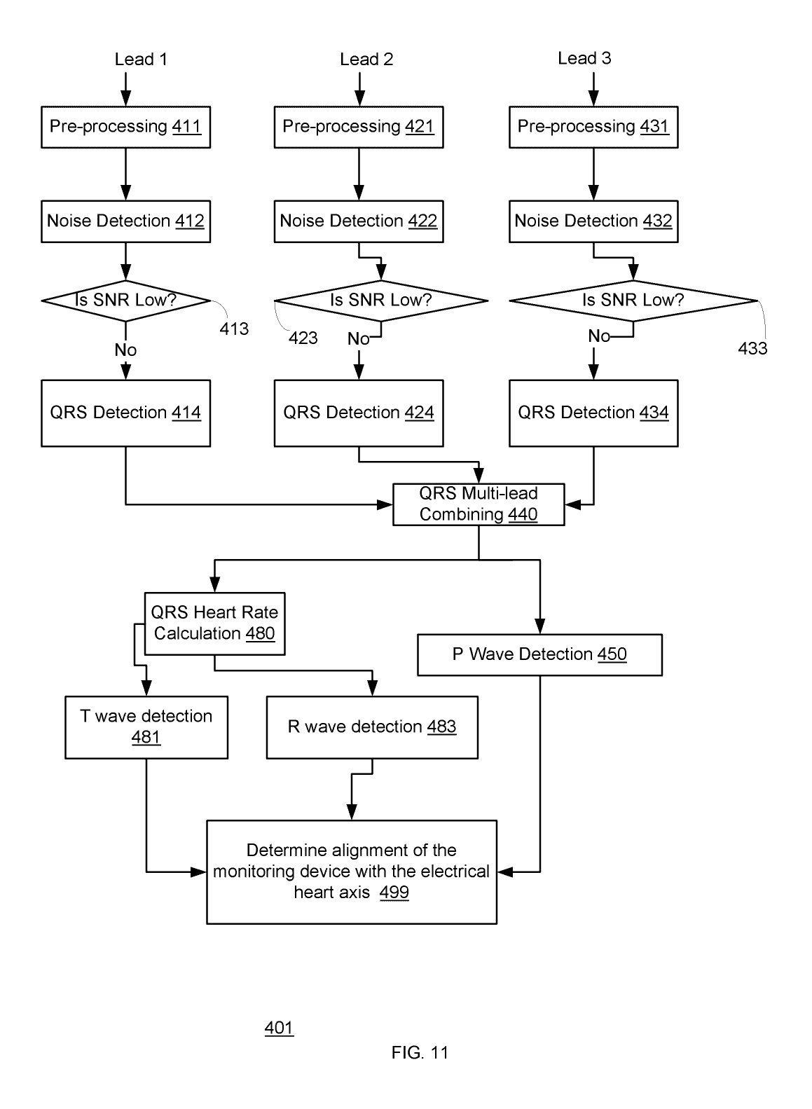

FIG. 11 illustrates a method 401 according to an embodiment of the invention.

Method 401 differs from method 400 by including steps 481, 483 and 499 instead of steps 460, 470, 482, 484, 486 and 490.

Method 401 starts by steps 411, 421 and 431 that are followed by steps 412, 422 and 432 that in turn are followed by steps 413, 423 and 433 respectively. Steps 413, 423 and 433 are followed by steps 414, 424 and 434 respectively. Steps 414, 424 and 434 are followed by step 440. Step 440 is followed by steps 440 and 480. Step 480 is followed by step 481 of T wave detection and step 483 of R wave detection.

Steps 481, 450 and 483 are followed by step 499 of determining, whether the monitoring device is aligned with the electrical heart axis of the person.

Method 401 may be executed whenever step 24 of method 20 is executed.

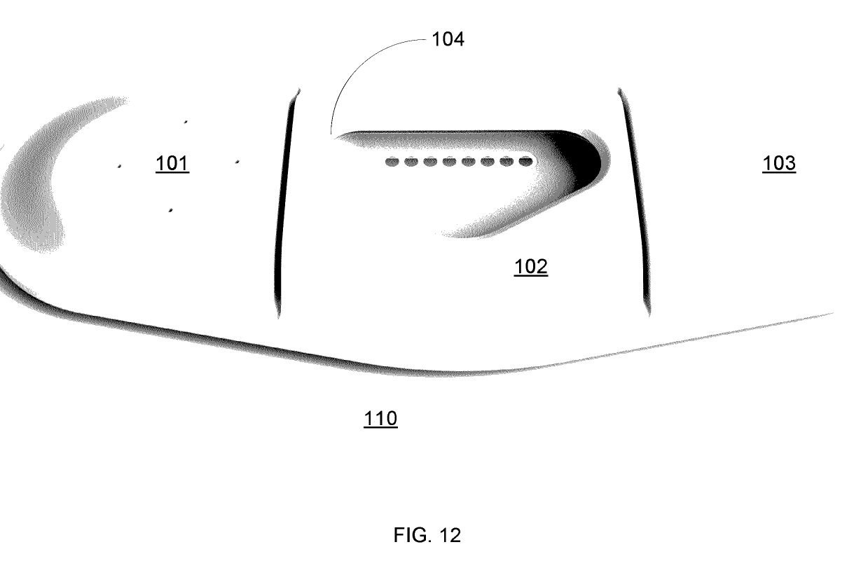

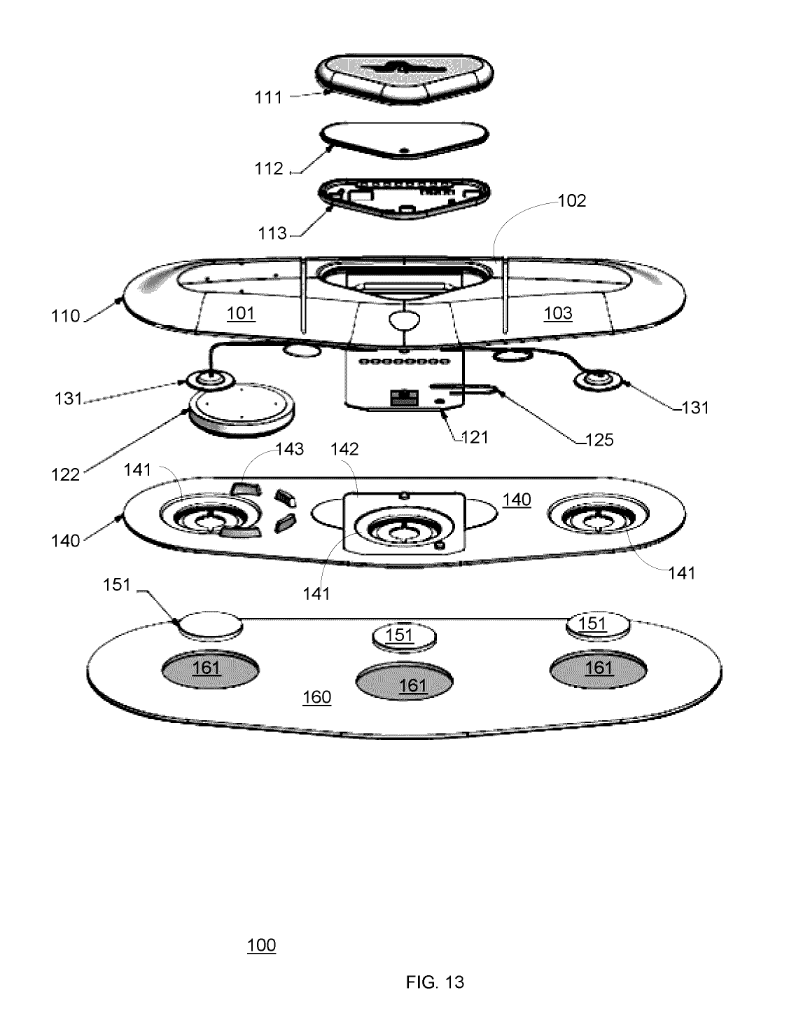

FIGS. 12 and 13 illustrates monitoring device 100 according to an embodiment of the invention.

Monitoring device 100 may include a top cover that includes a middle top cover part 102 that an opening 104. The middle top cover part 102 is positioned between left and right top cover parts 101 and 103 respectively.

Processing and transmitting module 110 may be pass through opening 104.

The processing and transmitting module 110 is removable and can be re-used multiple times while other parts of the monitoring device 100 are disposable.

The processing and transmitting module 110 includes a top cover 111, a bottom cover 113 and electronics (including a transceiver and a processor) 112 that are positioned between the top cover 111 and bottom cover 113.

The monitoring device 100 further includes a base layer 140 that is positioned above a bottom layer 160.

The bottom layer 160 may be flexible or not.

The bottom layer 160 includes three recesses 161.

Hydrogel elements 151 are positioned within the three recesses so that the bottom of the hydrogel elements 151 contacts the person when the bottom layer 160 is coupled to the person. It is noted that the bottom layer 160 may include adhesive elements for attaching the monitoring device 100 to the person.

The base layer 140 has three electrode housing 141, a battery interface 143 (for supporting and holding battery 122) and a processing and transmitting module interface 142 that contacts and supports the processing and transmitting module 110.

Base layer 140 is also connected to a printed circuit board 121 that may be connected to sockets (not shown).

The base layer 140 and especially the electrode housings 141 of the base layer 140 can be made of an elastic material such as but not limited to thermoplastic polyurethane (TPU). The TPU may be any polyurethane plastic.

Three electrodes 131 (out of which two electrodes are shown) are positioned within the three electrode housing 141 and their bottom is contacted by hydrogel elements 151. FIG. 13 illustrates three cables (including cable 125) that couples the printed circuit board 121 to the three electrodes.

The three electrodes 131 are used to provide for 1 lead arrhythmia detection.

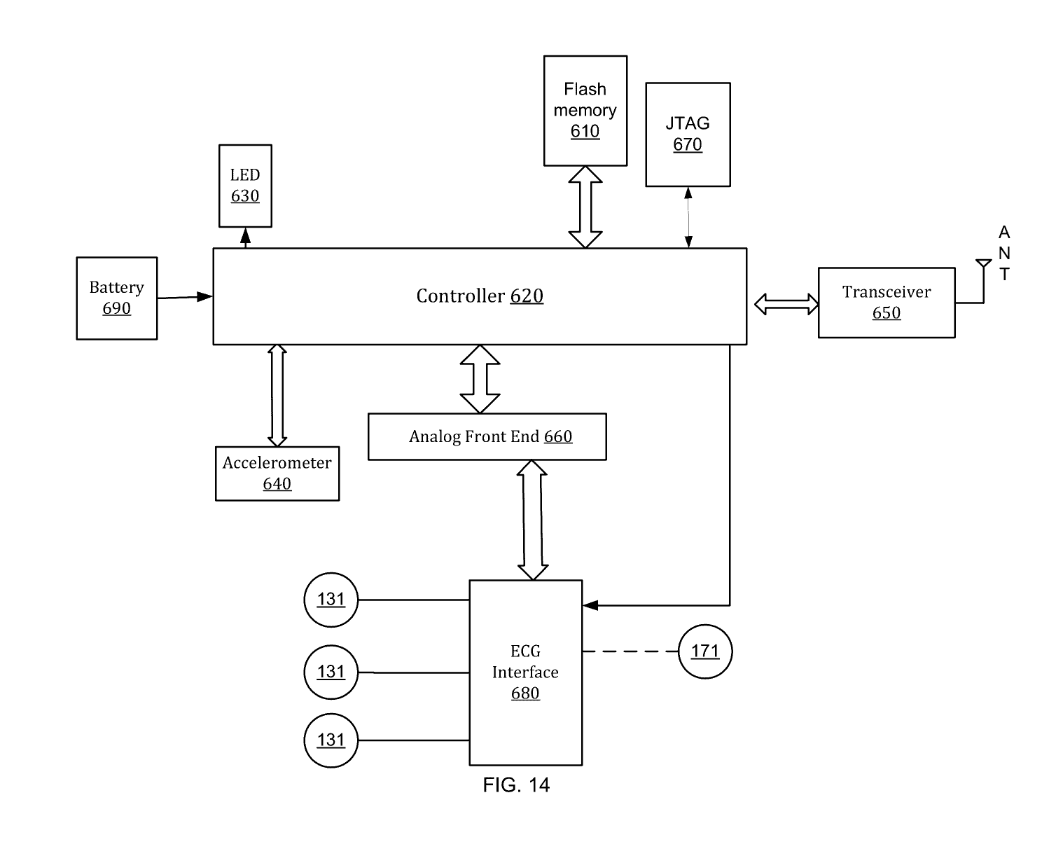

FIG. 14 is a block diagram of the monitoring device 100 according to an embodiment of the invention.

The processing and transmitting module 110 is illustrates as including a controller 620 and a transceiver 650 that is coupled between the controller 620 and an antenna. The processing and transmitting module 110 is detachable.

The controller 620 is configured to control the monitoring device 100 and may be configured to perform pre-processing (such as filtering) of the physiological signals sensed by the monitoring device 100. The preprocessing may include digital and/or filtering. The detection signals from the electrodes (either pro-processed or not) are stored in flash memory 610 and transmitted by transceiver 650 to the computerized control unit. The flash memory 610 can store several hours of detection signals.

The controller 620 is coupled to a light emitting diode (LED) 630, to battery 690, to a memory unit such as flash memory 610, to a JTAG interface 670, to an accelerometer 640, to an analog front end 660 and to an ECG interface 680.

The controller 620 may monitor the status of the battery 690 and send status indications (including a battery empty status alert) to the computerized control unit 300.

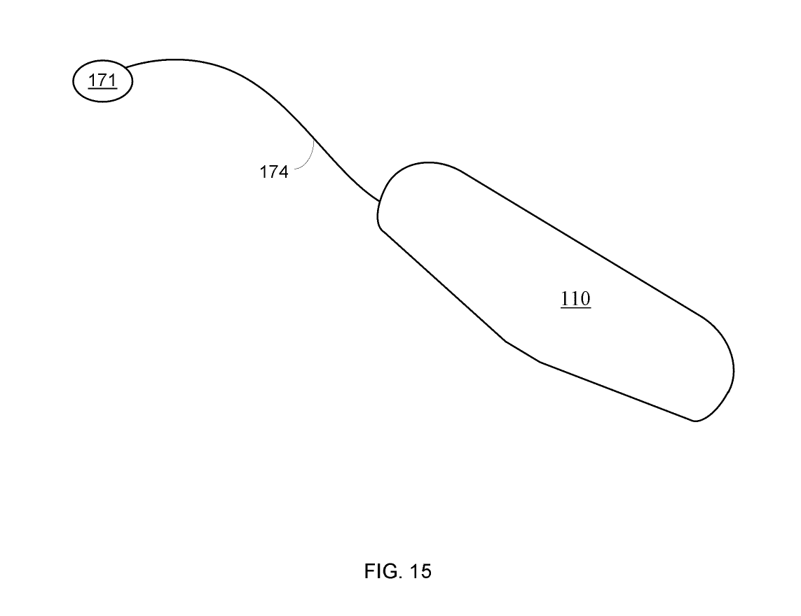

The ECG interface 680 is further coupled to electrodes 131 and to the analog front end 660. The analog front end 660 may also be coupled to an additional electrode 171 (also illustrated in FIG. 15).

The ECG interface 680 may provide the ECG signals from the electrodes 131 (and/or 171) and provide them to analog front end 660.

The analog front end 660 may process the ECG signals in the analog domain (for example—perform filtering operations) and perform an analog to digital conversion of the ECG signals to provide digital ECG signals. The analog front end 660 and/or the controller 620 may digitally processes digital ECG signals.

The analog front end 660 may be configured to test whether there is a proper connection with the electrodes 131 (for example by monitoring signals provided from the electrodes or lack of such signals or by sending a low current and testing the impedance) and to trigger the controller 620 to send an alert (to the computerized control unit 300) in case that one or more electrodes are not properly connected.

The JTAG interface 670 may be used for testing the controller 620 and/or for downloading firmware or software to the controller 620.

In case of communication failures error the monitoring device 100 may wait till the recovery of the communication and then transmit the ECG data that was gained during the failure period.

The monitoring device 100 and/or the computerized control unit 300 may monitor the person constantly, in a non-continuous manner, and the like.

FIG. 15 illustrates the monitoring device 100 having an additional electrode 171 according to an embodiment of the invention.

The additional electrode 171 is included in a housing (not shown in FIG. 15 but denoted 200 in FIG. 6). The additional electrode 171 is coupled to the ECG interface (not shown) within housing 110 via cable 174.

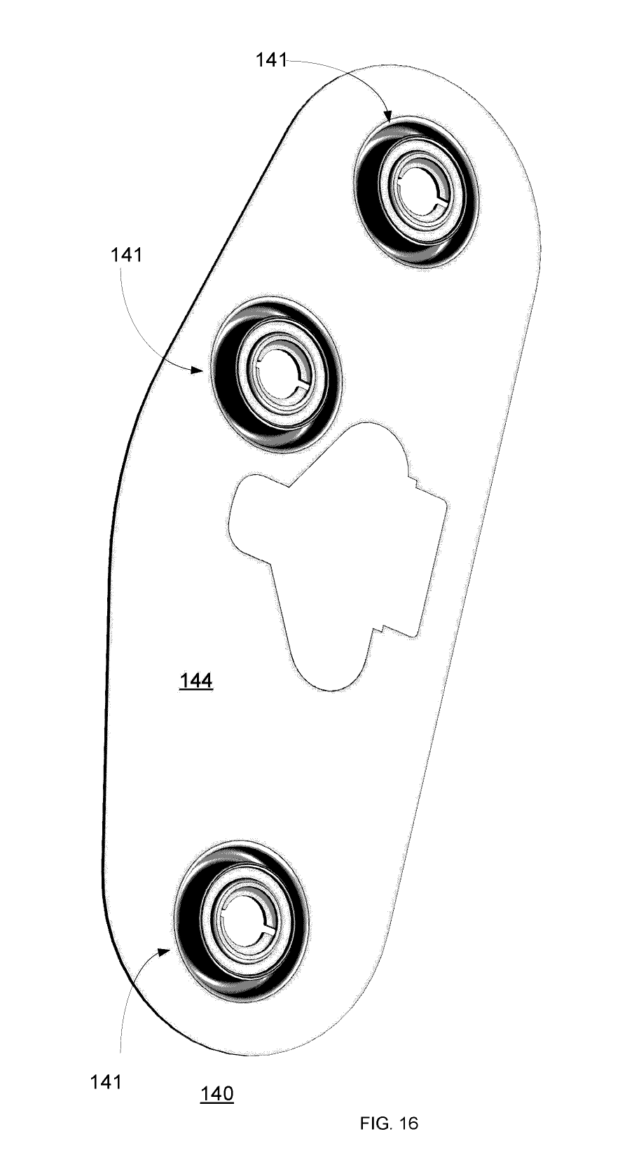

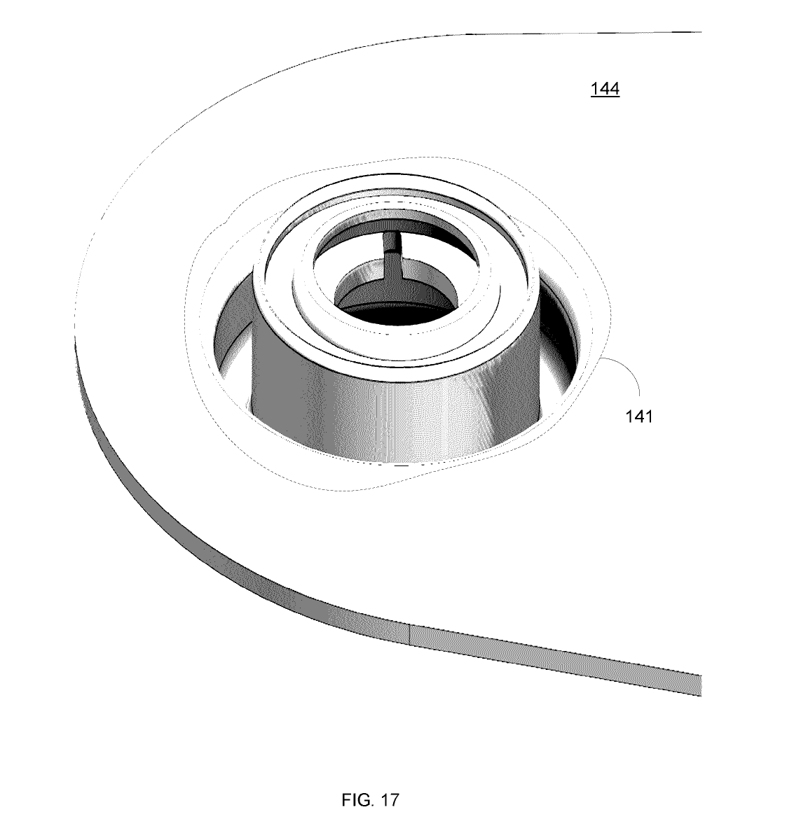

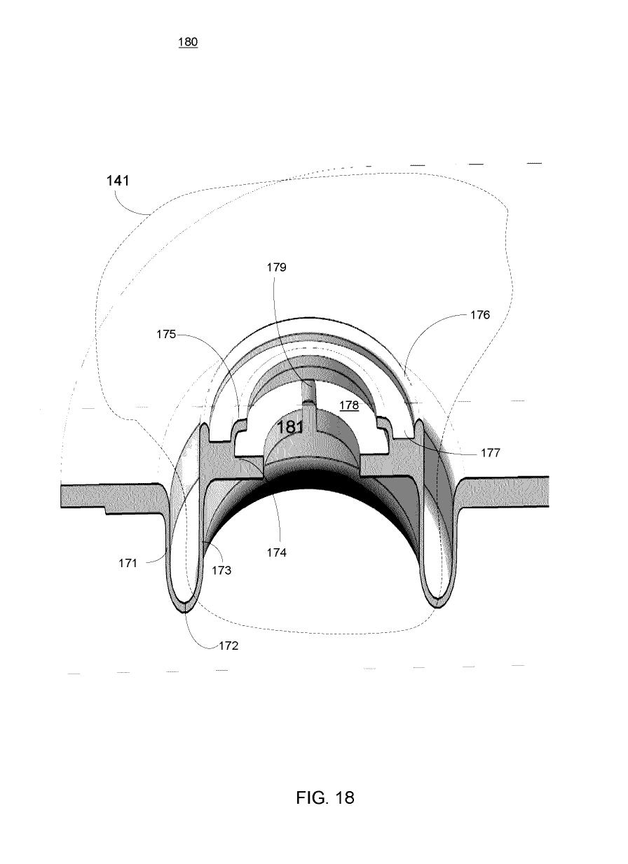

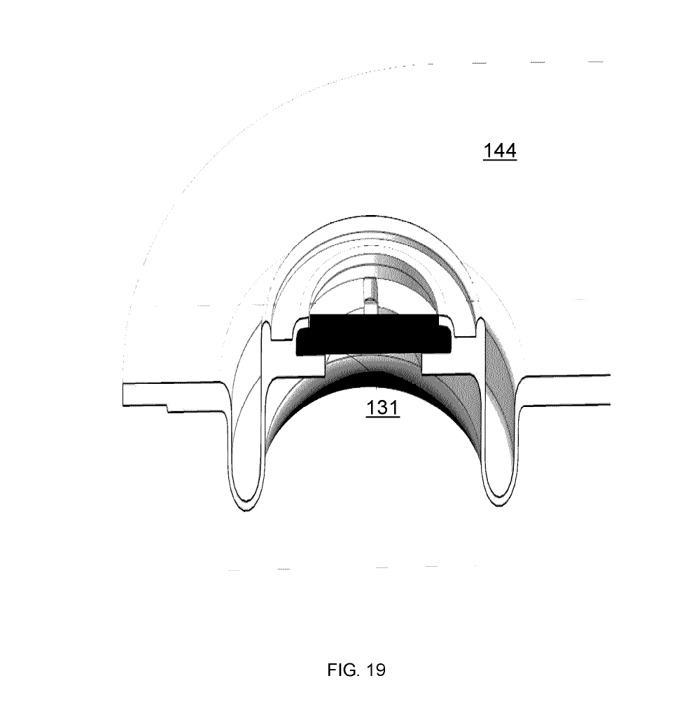

FIG. 16 illustrates base layer 140 of the monitoring device 100 according to an embodiment of the invention. FIG. 17 illustrates a portion of base layer 140 according to an embodiment of the invention. FIG. 18 is a cross sectional view of a portion of base layer 140 of the monitoring device according to an embodiment of the invention. FIG. 19 is a cross sectional view of a portion of base layer 140 and an electrode 131 according to an embodiment of the invention.

It has been found that rigidly connecting the electrode to the person introduces multiple noises—especially due to movements of the person, breathing of the person and the like.

In order to reduce these noises it has been found that suspending the electrode within an elastic housing without directly contacting the person reduces these noises dramatically.

The base layer includes a surface and one or multiple (for example three) electrode housings 141.

Each electrode housing 141 may include (a) a trench that includes an exterior sidewall 171, a curved bottom 172, an internal sidewall 173, and a group of structural elements that includes (b) a annular segmented base 174, (c) an inner annular cover 175, (d) an external ring 176, (e) an inner trench 177 that is delimited between the inner annular cover 175 and the external ring 176. The external ring 176 and internal sidewall 173 share a same facet.

It is noted that FIG. 18 illustrates the portion at an opposite orientation that the orientation of FIG. 13. Accordingly—external ring and inner annular cover 175 face the person while curved bottom 172 faces the battery 121 and the top cover 110.

The trench and the group of structural elements are integrated with each other—they can be made by a single mold.

The electrode is positioned within a cavity 178 that is formed by the annular segmented base 174 and the inner annular cover 175. The hydrogel element 151 is placed on the inner annular cover 175 and contacts electrode 132.

As illustrated above—the electrode housing 141 is made from elastic material and is virtually mechanically isolated from its surroundings it dumps mechanical noises that are introduced by movements of the person, person breathing and the like.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a non-transitory computer readable medium. All or some of the computer program may be provided on computer readable media permanently, removable or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as flash memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Although specific conductivity types or polarity of potentials have been described in the examples, it will be appreciated that conductivity types and polarities of potentials may be reversed.

Each signal described herein may be designed as positive or negative logic. In the case of a negative logic signal, the signal is active low where the logically true state corresponds to a logic level zero. In the case of a positive logic signal, the signal is active high where the logically true state corresponds to a logic level one. Note that any of the signals described herein may be designed as either negative or positive logic signals. Therefore, in alternate embodiments, those signals described as positive logic signals may be implemented as negative logic signals, and those signals described as negative logic signals may be implemented as positive logic signals.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. An electrode housing that is made of an elastic material and comprises:
a trench that includes an exterior sidewall, a bottom and an internal sidewall;
a group of structural elements;
wherein the internal sidewall is coupled to the group of structural elements;
wherein the group of structural elements is surrounded by the trench and defines a cavity that matches a shape and size of an electrode.

2. The electrode housing according to claim 1 wherein the trench and the group of structural elements are radially symmetric.

3. The electrode housing according to claim 1 wherein the group of structural elements comprise an annular segmented base and an inner annular cover that define the cavity.

4. The electrode housing according to claim 3 wherein the group of structural elements further comprise an external ring and an inner trench that is delimited between the inner annular cover and the external ring.

5. The electrode housing according to claim 3 wherein the annular segmented base defines an opening that is shaped and sized to match an upper part of an electrode.

6. The electrode according to claim 1 wherein the elastic material is a thermoplastic polyurethane.

7. A monitoring device, comprising: a detachable processing and transmitting module, multiple electrodes for sensing electrocardiogram signals; an elastic layer that comprises multiple electrode housings for supporting the multiple electrodes; wherein each electrode housing that is made of an elastic material and comprises a trench that includes an exterior sidewall, a bottom and an internal sidewall; a group of structural elements; wherein the internal sidewall is coupled to the group of structural elements;
wherein the group of structural elements is surrounded by the trench and defines a cavity that matches a shape and size of an electrode.

8. The monitoring device according to claim 7 wherein the trench and the group of structural elements are radially symmetric.

9. The monitoring device according to claim 7 wherein the group of structural elements comprise an annular segmented base and an inner annular cover that define the upper cavity.

10. The monitoring device according to claim 9 wherein the group of structural elements further comprise an external ring and an inner trench that is delimited between the inner annular cover and the external ring.

11. The monitoring device according to claim 9 wherein the annular segmented base defines an opening that is shaped and sized to match an upper part of the electrode.

12. The monitoring device according to claim 7 wherein the elastic material is a thermoplastic polyurethane.
